(19) United States
(12) Patent Application Publication
(10) Pub. No.: US 2020/0225239 A1
(43) Pub. Date: Jul. 16, 2020

(54) TREATMENT METHODS FOR MINIMAL RESIDUAL DISEASE

(71) Applicants: Massachusetts Institute of Technology, Cambridge, MA (US); Dana-Farber Cancer Institute, Inc., Boston, MA (US)

(72) Inventors: David Weinstock, Jamaica Plain, MA (US); Scott R. Manalis, Portland, OR (US); Mark Murakami, Cambridge, MA (US)

(73) Assignees: Massachusetts Institute of Technology, Cambridge, MA (US); Dana-Farber Cancer Institute, Inc., Boston, MA (US)

(21) Appl. No.: 16/739,778

(22) Filed: Jan. 10, 2020

Related U.S. Application Data
(60) Provisional application No. 62/790,742, filed on Jan. 10, 2019.

(51) Int. Cl. G01N 33/574; G01N 33/50

(52) U.S. Cl. CPC G01N 33/57488; G01N 2500/10; G01N 33/5011

(57) ABSTRACT

Patient samples are monitored to detect minimal residual disease (MRD) post successful cancer treatment. Upon detection of MRD, functional assays can be performed on living cancer cells from the patient to evaluate possibly effective therapies along with subsequent genomic or other more destructive assays to provide additional efficacy information using a single sample. An effective treatment against the MRD can be identified and selected for the patient. The patient can be monitored and the process repeated until MRD can no longer be detected.

### RELATED APPLICATION

This application claims priority under 35 U.S.C. § 119(e) to U.S. Provisional Application Ser. No. 62/790,742, filed Jan. 10, 2019, the entire contents of which are incorporated by reference herein.

### TECHNICAL FIELD

The disclosure relates to the detection and treatment of minimal residual disease.

### BACKGROUND

Many cancer chemotherapies result in significant reduction, or even elimination, of disease. Unfortunately, even after apparent remission, many patients retain minimal residual disease (MRD), which can result in relapse. Complete remission, however achieved, typically requires greater than 99% reduction in tumor burden. Accordingly, even with complete remission, some tumor cells remain. Those remaining tumor cells have traditionally been called minimal residual disease (MRD). While not all MRD cells contribute to a clinical relapse, some MRD cells can drive recurrence of a patient's cancer and, after relapse, the cancer may prove resistant to previously effective treatments leading to poor treatment outcomes and death. However, most clinicians wait until clinical relapse occurs to initiate additional or different therapy.

### SUMMARY

The invention provides methods for treating minimal residual disease in a patient after treatment. Devices and methods of the invention are used to test samples from a patient for live, malignant cells that exhibit characteristics of minimal residual disease. When live cells in a patient sample show the presence of minimal residual disease, the invention is further used for in vitro drug testing and therapeutic selection. The in vitro tests of the invention are used to determine what drugs effectively kill those cancer cells. Based on the test results, a drug is selected and given to the patient. The drug kills the cancer cells, preventing them from driving recurrence of the patient's cancer thereby preventing a relapse.

Once the cancer cells have been discovered and treated, the process can be performed again to detect and treat any subsequent round of minimal residual disease. In fact, methods of the invention may be performed iteratively, over time. After each course of treatment, a test is performed detect cancer cells indicative of minimal residual disease. Again, when cancer cells are detected, in vitro drug efficacy tests are performed for treatment selection. By providing an in vitro test useful for detecting minimal residual disease and therapeutic selection, methods of the invention prevent relapse after a successful cancer treatment. By performing the detection and therapeutic selection iteratively over successive rounds of treatment, minimal residual disease is changed from a condition that threatens full relapse to a condition of maintenance. By taking the threat of complete relapse out of minimal residual disease and changing it into a condition requiring only maintenance treatment, quality of life is vastly improved for cancer patients and life expectancy is greatly increased.

In certain embodiments, measurements are made using an instrument that includes a suspended microchannel resonator. Measuring functional properties of living cells in a sample from a patient can reveal the presence of cancer cells, thereby detecting minimal residual disease. For example, cancer cells continue to grow and accumulate mass. A suspended microchannel resonator can detect mass changes in living cells with great precision. By measuring mass change of individual living cells from a patient sample, the invention can detect minimal residual disease. By measuring such functional features of living cells in the presence of various therapeutics, the effects of those therapeutics on the cells can be observed to identify recommended treatments and to compare the efficacy of different possible treatments.

By analyzing changes in measurements such as mass accumulation of the patient's living cancer cells in the presence of various therapeutics, methods of the invention can be used to assess their efficacy in treating a specific patient's MRD. Thus embodiments of the disclosure include determining therapeutic susceptibility of cancer cells through monitoring of mass-accumulation using suspended microchannel resonators. Because cancers reflect a patient's personal genomics and involve rapid mutational development, each patient's cancer is unique and can respond differently to different treatments. Accordingly, direct efficacy analysis against the patient's own cancer cells provides useful insight in identifying, prescribing, and developing successful therapies.

An advantage of functional measurements using methods of the invention on living cells is that the cell is maintained for subsequent analysis. Accordingly, in addition to functional measurements such as mass accumulation measured by suspended microchannel resonators, secondary assays such as molecular analyses of cancer proteins or nucleic acids can be conducted to provide more detail regarding the patient's cancer. Exemplary assays include gene expression, sequencing analysis, For example, additional assays may include genome sequencing, single cell transcriptomics, single cell proteomics, or single cell metabolomics. When using other, destructive assays, additional analyses may require additional samples that may be difficult to obtain, especially when invasive or painful biopsies are required or where sample is limited as may often be the case when dealing with MRD levels of cancer. Accordingly, the ability of the functional measurement techniques described herein to leave intact cells for subsequent analysis allows for more detailed characterization of a patient's cancer in an efficient, less expensive, and less invasive manner.

Additionally, systems of the invention, especially when using suspended microchannel resonator analysis, can incorporate the additional analyses into a single microfluidic system. Microfluidic sequencing, flow cytometry, microarrays, and other analysis systems are well known and can be integrated into a microfluidic system downstream of the suspended microchannel resonator array in a single or modular system with exchangeable components depending on the secondary analysis to be performed.

Systems and methods of the invention can be performed on a variety of patient samples including cancer cells isolated from patient bodily fluids (e.g., mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CS) or tissue samples (e.g., fine need biopsies). Where tissue samples are obtained, systems and methods of the invention may include tissue disaggregation steps in order to isolate single cells for functional analysis and other assays described herein.

In certain embodiments, after detection of MRD but before performing efficacy assays, a database including therapeutic, patient, and outcome information can be queried to determine a subset of possibly effective therapies based on certain criteria and past results and patterns in the data. The results of such an in silico analysis can then be used to populate the therapeutics for the initial efficacy assays. The database can be updated with each new patient's information and outcome. Machine learning analysis can be used to identify hidden correlations between patient data and responsiveness to various therapies. Results of the efficacy assays can be fed back into the database along with information about the patient and their cancer cells as well as eventual treatment outcomes for the patient to refine the in silico analysis for future queries.

Database information used in the in silico analysis can include drug information such as toxicology, past efficacy, pharmacokinetics, and cost. Many features can be tied to specific patients (with associated patient and outcome information) or may be determined generally or through statistical analysis. Patient information may include, for example, genetic information, past treatments, age, gender, medical history, family history, and health measurements (e.g., weight, height, body mass index, blood pressure, cholesterol, and blood sugar). Outcomes with selected treatments can be tracked and linked to the above patient and drug information to build a more robust database and identify new links between patient or drug features and expected outcomes through machine learning analysis. Machine learning analysis can include for example, a random forest, a support vector machine (SVM), or a boosting algorithm (e.g., adaptive boosting (AdaBoost), gradient boost method (GSM), or extreme gradient boost methods (XGBoost)), or neural networks such as H2O.

After each successful iterative treatment, the patient can be monitored to detect MRD and repeat the process, with potentially different effective treatments identified and used each time. The process can continue until no MRD can be detected in the patient.

Monitoring patients for MRD and treating before clinical relapse offers a number of advantages. First, as cancer proliferates into the number of cells necessary for clinical relapse, clonal complexity also increases providing an increased likelihood of sub-clonal resistance to one or more therapeutics. Accordingly therapies that may have proven effective at treating the MRD may no longer work on the more developed recurrent cancer. Additionally, more substantial and organized numbers of recurrent cancer cells can remodel microenvironments and reprogram infiltrating hematopoietic cells, thereby lowering effectiveness of treatments that may have worked on MRD levels of cancer cells. Also, patients in remission having MRD but not yet in full relapse, are generally healthier and more tolerant of certain treatments and side-effects, again increasing the number of available treatment options if used at the MRD stage. Finally, there are simply less cancer cells to eradicate at the MRD stage meaning a chance of complete eradication and cure is greater at that stage than upon clinical relapse.

Aspects of the invention include a method for treating minimal residual disease. A sample is obtained from a patient after an effective treatment for cancer and a first assay is conducted on the sample to detect minimal residual disease. An efficacy assay is performed to determine efficacy of a plurality of candidate therapies in treating the minimal residual disease and an effective next treatment is selected for the patient from the plurality of candidate therapies. The first assay may measure a functional feature of cells in the sample. The efficacy assay can be performed in cancer cells obtained from the patient and the cells may be live. Methods of the invention may further include disaggregating the cells from a tissue sample before performing the efficacy assay. The efficacy assay can include measuring a change in mass in the live cells.

In certain embodiments, change in mass may be measured using a suspended microchannel resonator instrument. The suspended microchannel resonator instrument can include an array of suspended microchannel resonators. The suspended microchannel resonator instrument may flow living cells through the array of suspended microchannel resonators. The suspended microchannel resonator instrument can include a pressure control system operable to actively adjust fluidic pressure within the system to load living cells, one at a time, into a suspended microchannel resonator. The efficacy assay may include a functional measurement of live cells in the presence of one of the plurality of candidate treatments. The functional measurement can include measuring a change in mass in the live cells.

Methods of the invention may further comprise administering the effective next treatment to the patient and repeating the conducting, performing, selecting and administering steps until the functional feature is not detected in the first assay.

### BRIEF DESCRIPTION OF THE DRAWINGS

FIG. 1 diagrams a method for detecting and treating minimal residual disease.

FIG. 2 illustrates performing an assay on a sample.

FIG. 3 shows a suspended microchannel resonator (SMR) device.

FIG. 4 shows a serial suspended microchannel resonator (sSMR) array.

FIG. 5 is a diagram of an exemplary SMR detection system.

FIG. 6 diagrams a sequencing workflow consistent with the present disclosure.

FIG. 7 is a block diagram of a system consistent with the present disclosure.

### DETAILED DESCRIPTION

Systems and methods of the invention provide tools for monitoring and iteratively treating minimal residual disease (MRD) after a successful initial treatment of a patient's cancer. MRD can be detected in one or more tissue or body fluid samples from a patient at which point, cancer cells from the patient sample can be assayed against one or more potential therapies to determine the most effective next treatment. By detecting and treating MRD before clinical relapse, patient prognoses are improved and the chance of recurrence is reduced. For example, once a primary tumor has been successfully treated, the remaining pockets of MRD may be dispersed. MRD including independent metastases derived from the same carcinoma can undergo divergent evolution that leads to differential therapeutic responses. Systems and methods of the invention account for these variations by iteratively testing and implementing different therapies. Accordingly, even if a patient retains heterogenic pockets of MRD having different therapeutic responses, an effective treatment for each can be identified through the iterative testing contemplated herein.

Cancer describes several diseases characterized by abnormal cell growth capable of spreading to other parts of the body. Types of cancer are characterized by the cells from which they originate. Cancer types include carcinomas such as breast, prostate, lung, pancreatic, and colon cancers that arise from epithelial cells. Sarcomas are derived from connective tissue (e.g., bone, cartilage, fat, or nerve cells). Lymphoma and leukemia arise from hematopoietic cells and are found in the lymph nodes and blood of afflicted patients. Cancer of plasma cells (myeloma) is another cancer found in blood. Germ cell cancers derived from pluripotent cells and blastomas from precursor cells or embryonic tissue are other types of cancer. Systems and methods of the invention relate to identifying treatments for any of the above cancer types including both those readily detectable in body fluids (e.g., lymphoma, leukemia, or multiple myeloma) as well as solid tumors (e.g., carcinomas).

One measure of a successful cancer treatment is complete remission, usually defined as the absence of visible tumor using medical imaging techniques or possibly histological examination of tissue. However, fully-transformed, malignant cells that remain in a patient who achieves complete remission may be referred to as minimal residual disease (MRD). See, Luskin, et al., 2018, Targeting minimal residual disease: a path to cure?, Nat. Rev. Cancer, 18:255-263, incorporated herein by reference. Those MRD cells can be further classified based on their capacity to proliferate into a diagnosable relapse. MRD cells incapable of proliferation may have been damaged through previous treatments or may have differentiated beyond the ability to act as cancer stem cells. MRD cells that retain the capacity to proliferate have properties consistent with malignancy in that they are capable of proliferating into a clinical relapse and have fully transformed. The invention provides devices and methods for the in vitro identification of MRD cells that have the capacity to proliferate into a diagnosable relapse by making in vitro measurements of functional properties of individual living cells from a sample obtained from a patient. Functional properties that may be hallmarks of the capacity to proliferate into a diagnosable relapse include growth, which may be exhibited ex vivo by an individual living cell as mass change or a positive non-zero mass accumulation rate.

Samples can be obtained from patients in remission from cancers of any type including myeloma, lymphoma, and leukemia (having detectable MRD cells in bone marrow or easily sampled bodily fluids) as well as solid tumors conventionally thought too difficult to sample for. See Luskin, at 257.

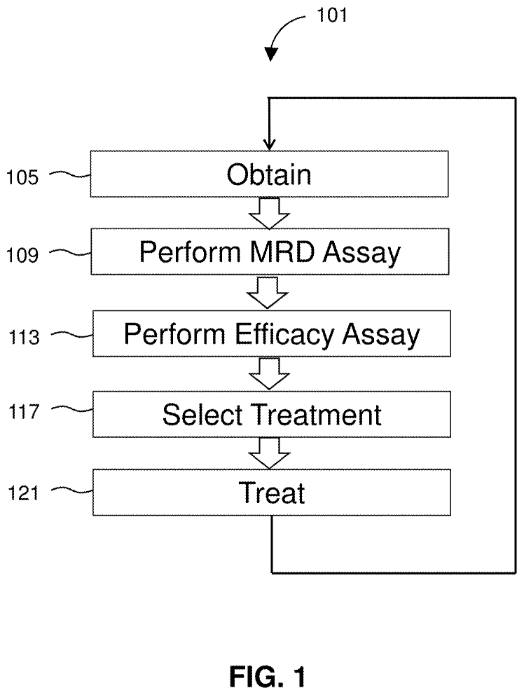

FIG. 1 shows steps of a method 101 for detecting and treating MRD. The method 101 includes obtaining 105 a sample from a patient after an effective treatment. The method 101 then includes performing 109, on the sample, an assay to detect MRD. As noted below samples may include, for example, body fluid or tissue biopsies. MRD assays may be cancer-dependent. For example, blood-based assays (e.g., circulating cell free DNA analysis) or bone marrow samples may be particularly useful for MRD analysis in leukemia. Blood or lymph may also be sampled to identify residual or metastatic tumor cells for solid tumors. In other embodiments, imaging and/or tissue biopsies of the liver, bone, spleen, lung can be analyzed to detect MRD from solid tumors. Apheresis can be used to provide more sensitive results to identify MRD with low levels of circulating cells or cell-free nucleic acid. DNA sequencing, RNA sequencing, or immunological testing can all be used to detect and/or quantify MRD among healthy material in a patient sample by identifying residual nucleic acids or metabolites as described above.

As noted, MRD measurement has been characterized in leukemia including DNA-based tests used to detecting leukemia-specific DNA sequences using PCR and sequencing or microarrays. The markers used for DNA-based testing for MRD in leukemia include chromosomal translocations or targeting genes including microsatellites, immunoglobulin and T cell receptors. RNA-based tests are also used to detect leukemia-specific RNA sequence. CDNA can be reverse transcribed from the RNA followed by PCR amplification to produce a sufficient quantity of DNA to test. RNA-based tests can be beneficial when DNA tests prove impractical (e.g., where a leukemia-indicative translocation occurs over a large length of the chromosome). Patient-specific MRD detection using immunoglobulin or T cell receptors is another way of measuring MRD in leukemia. A leukemia-specific immunoglobulin or T cell receptor clone is amplified using PCR and the variable region sequenced. Sequence-specific PCR primers can then be designed that will only amplify the patient-specific clone, thereby indicating presence of MRD. Immunological-based testing of leukemia is another technique using proteins on the surface of the cells, stains, and antibodies thereto to label and detect leukemic cells via flow cytometry. Systems and methods of the invention contemplate using any of the above techniques to detect residual tumor cells or nucleic acids from solid tumors that have been shed into bodily fluids or may be present in biopsies or other tissue samples taken periodically after successful cancer treatment.

Upon detection of MRD in a patient, cells may be isolated from the same sample as for the first, MRD assay or from a subsequently obtained sample for an efficacy assay. An efficacy assay is performed 113 to determine therapeutic susceptibility of cancer cells to one or more therapeutics. Determining therapeutic susceptibility of cancer cells through monitoring of mass-accumulation using suspended microchannel resonators may include techniques described in Cetin, 2017, Determining therapeutic susceptibility in multiple myeloma by single-cell mass accumulation, Nat Com, 8, 1613, incorporated by reference. Because cancers reflect a patient's personal genomics and involve rapid mutational development, each patient's cancer is unique and can respond differently to different treatments.

One or more live cells are isolated from a biological sample of a patient suspected of having MRD. A biological sample may include a human tissue or bodily fluid and may be collected in any clinically acceptable manner. A tissue is a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CS. A sample also may be media containing cells or biological material. A sample may also be a blood clot, for example, a blood clot that has been obtained from whole blood after the serum has been removed. In certain embodiments, the sample is blood, saliva, or semen collected from the subject.

Examples of biopsies that may provide cells for analysis using systems and methods described herein can include, needle biopsy, bone biopsy, bone marrow biopsy, liver biopsy, kidney biopsy, aspiration biopsy, prostate biopsy, skin biopsy, or surgical biopsy. Systems and methods of the invention should be performed using living cells. Accordingly, cells should be preserved in culture media or otherwise stored in a manner to minimize cell death. In order to facilitate a living sample, systems and methods of the invention can be applied to samples within less than about 1 hour, 6 hours, 12 hours, 24 hours, 36 hours, or 48 hours of obtaining the sample.

Samples can be obtained 105 by excising tissue or cells from a patient through, for example, a blood draw or biopsy. In certain embodiments, obtaining 105 a sample may include receiving a sample at, for example, a remote laboratory. The sample may have been taken by a medical professional at a hospital or other medical center and packaged and transported to the laboratory for analysis. Obtaining the sample may include opening the packaged sample before conducting an analysis thereof.

The isolation of the one or more live cells from the biological sample may be performed via any known isolation techniques and methods for maintaining a viable collection of cells, which may include one or cancer and/or cancer-related immune cells (e.g., lymphocytes includes T-cells and/or B-cells). For example, if the sample is a tissue sample from a tumor or growth suspected of being cancerous, the tissue sample may undergo any known cell isolation, separation, or dissociation techniques which may involve physical methods (i.e., use of mechanical force to break apart cellular adhesions) and/or reagent-based methods (i.e., use of fluid mediums to break apart cellular adhesions). For example, in one embodiment, a tissue sample (i.e., a fine needle aspirate from a tumor) may be disaggregated to produce a suspension of individual live cells to allow for analysis of cells independently. Cells may be isolated from patient bodily fluids or tissue samples. Where tissue samples are obtained, systems and methods of the invention may include tissue disaggregation steps in order to isolate single cells for functional analysis and other assays described herein. The extracellular matrix of tissue samples must be broken down to recover single cells. Many procedures for dissociating solid tumors are known as described, for example, in Cunningham, 1999, Tissue Disaggregation, Methods in Molecular Biology, 115:257-60, incorporated by reference. Disaggregation methods include one or more mechanical, enzymatic, or chemical manipulations. The tissue sample may undergo initial disaggregation by way of application of a physical force alone to break the tissue sample into smaller pieces, at which point the sample may be exposed to proteolytic enzymes that digest cellular adhesion molecules and/or the underlying extracellular matrix to thereby provide single cells within a suspension.

In a preferred embodiment, the obtaining step 105 includes drawing the sample from a solid tumor by fine needles aspiration. A solid tumor may interrogated via fine needle aspiration to retrieve a cell mass, or tissue sample, that includes cancer cells. Methods may include using a needle, such as a fine-needle aspiration biopsy using a sharp 25-gauge, 1-inch long needle. A suitable needle is the sharp 25-gauge, 1-inch long needle sold under the trademark PRECISION GLUIDE by BD (Franklin Lakes, N.J.). The needle may be attached to a 10 ml aspirating syringe.

The biopsy needle may be passed into a lesion or tumor. Once the tip of the needle is advanced into the lesion, the tumor cells are aspirated. The plunger of the syringe may be pulled and released a few times, allowing the suction force to equilibrate. The needle is withdrawn and a tissue sample or clump of cells is deposited in or on a substrate such as a slide, culture dish, membrane, or other material. In some embodiments, the clump of cells is deposited on a surface within a collection tube or flask, such as a 1.5 mL microcentrifuge tube sold under the trademark EPPENDORF. Each aspirate may be flushed into the flask using culture media, saline, or a maintenance/nutrient media. The aspiration material may be filtered to deposit clumps or samples of tissue on the surface of a filter membrane. The cell mass may be deposited, e.g., on a nitrocellulose membrane and disaggregated using, e.g., proteases such as collagenase and/or displace. Live cells may be washed into a fluidic tube or system with and supported by a suitable media such as a Ham's nutrient mixture. For information, see Rajer, 2005, Quantitative analysis of fine needle aspiration biopsy samples, Radiol Oncol 39(4):269-72, incorporated by reference.

The tissue sample or clump of cells is disaggregated. Any suitable technique may be used to disaggregate the tissue sample/clump of cells. For example, disaggregation may include physical or mechanical disaggregation, chemical disaggregation, proteolytic disaggregation, or any combination thereof. In some embodiments, proteolytic disaggregation is performed using one or more enzymes. Any suitable enzymes may be used. In some embodiments, the tissue sample/clump of cells is washed with and digested by collagenase I and dispase II. The resultant free cells may be held in a suitable nutrient media such as, for example, Ham's F12 Kaighn's Modification medium in presence of 1 mU/mL bovine thyrotropin (TSH), 10 μg/mL human insulin, 6 μg/mL transferrin, and 10-8 M hydrocortisone.

Thus the method 101 may include obtaining 105 a fine needle aspirate tissue sample, from a solid tumor, that includes live cancer cells that are disaggregated (preferably by proteolytic techniques) from any tissue or clump so that individual live cells may be separately addressed, e.g., subjected to a measurement of some functional property of those cells. It should be noted that the reagents selected for assisting in the disaggregating step should keep the cells intact and not kill the cells.

Other methods currently used for single cell isolation include, but are not limited to, serial dilution, micromanipulation, laser capture microdissection, FACS, microfluidics, Dielectrophoretic digital sorting, manual picking, and Raman tweezers. Manual single cell picking is a method is where cells in a suspension are viewed under a microscope, and individually picked using a micropipette, while Raman tweezers is a technique where Raman spectroscopy is combined with optical tweezers, which uses a laser beam to trap, and manipulate cells. Dielectrophoretic (DEP) digital sorting method utilizes a semiconductor controlled array of electrodes in a microfluidic chip to trap single cells in DEP cages, where cell identification is ensured by the combination of fluorescent markers with image observation and delivery is ensured by the semiconductor controlled motion of DEP cages in the flow cell.

After obtaining MRD cells from a patient sample, the cells can be assayed 113 to determine efficacy of various therapeutics. The selected therapeutics for efficacy assays may be determined through an in silico analysis in certain embodiments as detailed below. Efficacy assay(s) can include measuring a functional cancer biomarker in the one or more live cells. For example, in one embodiment, the functional cancer biomarker includes mass and/or mass change of the one or more live cells in the presence of one or more of the identified therapeutics. In some embodiments, as will be described in greater detail herein, a first efficacy assay involves loading individual live cells into a measurement instrument and flowing the live cells through the measurement instrument. The measurement instrument may generally include a microfluidic platform capable of direct measurement of single-cell mass and growth rate. The measurement instrument may make use of a suspended microchannel resonator. Suspended microchannel resonators are described in Cermak, 2016, High-throughput measurement of single-cell growth rates using serial microfluidic mass sensor arrays, Nat Biotechnol, 34(10):1052-1059, incorporated by reference. Upon flowing the live cells through the measurement instrument, a functional cancer biomarker in the one or more live cells is obtained, the functional cancer biomarker including mass or mass accumulation rate (MAR). The live cells remaining in a living state upon passing through the measurement instrument, such that they are accessible for one or more additional live cell assays downstream from the first assay. Additional assays can be performed on the cells in combination with the functional measurement and can include genome sequencing, single cell transcriptomics, single cell proteomics, and single cell metabolomics.

The method 101 further includes selecting 117 one or more of the identified therapeutics for treating the patient's cancer where the determined efficacy is above a certain threshold. In certain embodiments, the therapy may be selected by comparing the determined efficacies of all measured therapeutics that were identified in step 109 and selecting the most effective. The method 101 can further include treating 121 the patient using the selected therapeutic. After treatment, the process can repeat, with another sample being obtained 105 and so on until MRD can no longer be detected.

Methods can include in silico analysis before the efficacy assay to determine one or more therapeutics that meet predetermined criteria relating to toxicology, efficacy, pharmacokinetics, side-effects, drug interactions, patient compliance, or cost. The predetermined criteria may be preset for general patients or classes of patients or may be patient-specific. For example, patients in a certain age group may have lower thresholds for toxicology and patients in a certain economic group (or without insurance coverage) may have a lower tolerance for cost. In such cases, patient information may be supplied to determine the thresholds. Patient information may include genetic information, past treatments, age, gender, medical history, family history, and health measurements (e.g., weight, height, body mass index, blood pressure, cholesterol, and blood sugar), race, income, insurance status and ability to pay, environmental or geographic information and history (e.g., place of birth and historic and current places of residence). Patient information may be obtained directly from the patient or individuals associated with the patient through, for example, a questionnaire or an interview. The information can also be obtained from a medical professional after consultation with the patient. In certain embodiments, patient information may be obtained, along with a patient sample, by a medical professional and transmitted (physically or electronically) to a remote facility where the in silico analysis can be conducted and the sample can be processed.

Once the predetermined thresholds are determined and entered, the in silico analysis can proceed. The analysis can access a database of drug information to identify all of the therapies that meet each criterion. For example, a patient's ability to pay may correspond to a level of $1,000 per month. Accordingly, the database will be searched for drugs and therapies having a cost of $1,000 per month or less and the results will be examined based on the remaining criteria before a set of results consisting of therapeutics meeting each of the criteria thresholds is determined. The in silico analysis can result in a list of potential therapies being output (e.g., on a display or printed report).

Criteria such as toxicology, efficacy, side-effects, drug interactions, and patient compliance may be analyzed based on specific patient data using correlations identified through machine learning analysis as described below. Accordingly, patient information and any known cancer information may be provided for in silico analysis to predict toxicology, efficacy, side-effects, drug interactions, or patient compliance for a given therapy based on machine-learning-identified links between those parameters and the received patient or cancer information. The predicted toxicology, efficacy, side-effects, drug interactions, or patient compliance for a given therapy and patient can then be compared to the predetermined criteria to identify 109 a subset of therapeutics for further analysis. In such embodiments known patient or cancer information can include previously obtained genomic or proteomic data relating to the patient or their cancer in addition to the characteristics described below.

Machine learning analysis can be used to identify correlations between patient, cancer, MRD, and treatment characteristics and various treatment outcomes such that future analysis of patient data can provide a subset of treatment options likely to prove successful for that patient. Any machine learning algorithm may be used for the systems and methods described herein including, for example, a random forest, a support vector machine (SVM), or a boosting algorithm (e.g., adaptive boosting (AdaBoost), gradient boost method (GSM), or extreme gradient boost methods (XGBoost)), or neural networks such as H2O. Machine learning algorithms generally are of one of the following types: (1) bagging, (2) boosting, or (3) stacking. In bagging, multiple prediction models (generally of the same type) are constructed from subsets of classification data (classes and features) and then combined into a single classifier. Random Forest classifiers are of this type. In boosting, an initial prediction model is iteratively improved by examining prediction errors. Adaboost.M1 and eXtreme Gradient Boosting are of this type. In stacking models, multiple prediction models (generally of different types) are combined to form the final classifier. These methods are called ensemble methods. The fundamental or starting methods in the ensemble methods are often decision trees. Decision trees are non-parametric supervised learning methods that use simple decision rules to infer the classification from the features in the data. They have some advantages in that they are simple to understand and can be visualized as a tree starting at the root (usually a single node) and repeatedly branch to the leaves (multiple nodes) that are associated with the classification.

Random forests use decision tree learning, where a model is built that predicts the value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, L. Random Forests, Machine Learning 45:5-32 (2001), incorporated by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable.

SVMs can be used for classification and regression. When used for classification of new data into one of two categories, such as having a disease or not having a disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering. See Ben-Hur, A., et al., (2001), Support Vector Clustering, Journal of Machine Learning Research, 2:125-137.

Boosting algorithms are machine learning ensemble meta-algorithms for reducing bias and variance. Boosting is focused on turning weak learners into strong learners where a weak learner is defined to be a classifier which is only slightly correlated with the true classification while a strong learner is a classifier that is well-correlated with the true classification. Boosting algorithms consist of iteratively learning weak classifiers with respect to a distribution and adding them to a final strong classifier. The added classifiers are typically weighted in based on their accuracy. Boosting algorithms include AdaBoost, gradient boosting, and XGBoost. Freund, Yoav; Schapire, Robert E (1997). “A decision-theoretic generalization of on-line learning and an application to boosting”. Journal of Computer and System Sciences. 55: 119; S. A. Solla and T. K. Leen and K. Müller. Advances in Neural Information Processing Systems 12. MIT Press. pp. 512-518; Tianqi Chen and Carlos Guestrin. XGBoost: A Scalable Tree Boosting System. In 22nd SIGKDD Conference on Knowledge Discovery and Data Mining, 2016; the contents of each of which are incorporated herein by reference.

Machine learning algorithms can be trained on data sets useful for the intended purpose of the machine analysis. For example, to train for machine analysis of cancer cell features and patient data, a machine learning algorithm can be provided with a training data set including patient information, therapeutic information, cancer features, and associated outcomes for specific patient, therapeutic, and cancer combinations. The algorithm can then identify common patterns in the data that are indicative of an expected outcome. A particular advantage of machine learning algorithms is the ability to identify patterns that cannot be easily perceived by human analysis.

Considerations such as cost and availability of a given therapeutic may not impact drug effectiveness and, accordingly, may be omitted from machine learning analysis used to identify correlations between drug, patient, and cancer characteristics and treatment outcomes. Such considerations, as well as side-effects, drug interactions, and patient compliance can, however, be important considerations when determining a practical treatment for a given patient. Accordingly, that data may be included in subsequent application of the learned correlations in the in silico analysis determinations of recommended treatments for patient-specific efficacy trials.

Therapeutic information may include cost of the treatment, toxicology, pharmacokinetics, side effects, patient compliance, availability, drug interactions, and past efficacy. Many features can be tied to specific patients (with associated patient and outcome information) or may be determined generally or through statistical analysis. Patient information may include, for example, genetic information, past treatments, age, gender, medical history, family history, and health measurements (e.g., weight, height, body mass index, blood pressure, cholesterol, and blood sugar). Cancer information may include functional measurements and other assay-derived characteristics of the cancer cell and tissue samples as described below. Categories may overlap, for example, patient and drug information may both include a patient's past responsiveness to a particular drug. Outcomes may include, for example, complete or partial remission, a number of years lived after treatment, or a slowed disease progression. Outcomes with selected treatments can be tracked and linked to the above patient and drug information to build a more robust database and identify new links between patient or drug features and expected outcomes.

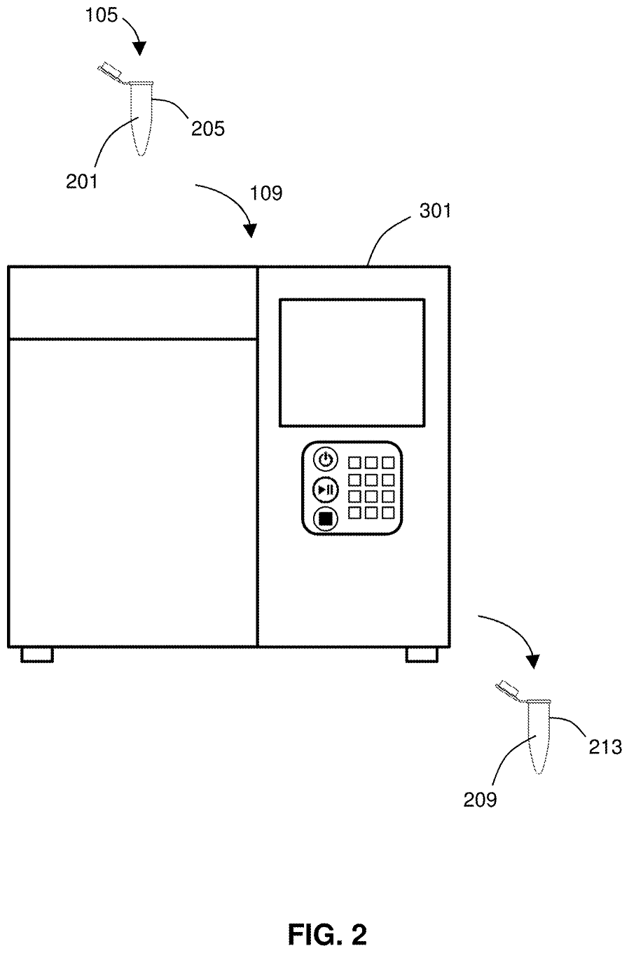

FIG. 2 shows a sample 201 provided within a suitable container 205, wherein the sample 201 includes one or more live cells including at least one of a cancer cell and a cancer-related immune cell obtained 105 from a patient known to have, or suspected of having, cancer. For example, in some embodiments, samples may be collected and stored in their own container, such as a centrifuge tube such as the 1.5 mL micro-centrifuge tube sold under the trademark EPPENDORF FLEX-TUBES by Eppendorf, Inc. (Enfield, Conn.).

FIG. 2 further illustrates the loading of the one or more live cells into an instrument 301 capable of performing 109 the first assay on the one or more live cells. The instrument 301 is used to measure a functional cancer biomarker in the one or more live cells, such as single-cell biophysical properties, including, but not limited to, mass, growth rate, and mass accumulation of an individual living cell. The initial assay may generally be performed with an instrument 301 comprising a suspended microchannel resonator (SMR). The SMR may be used to precisely measure biophysical properties, such as mass and mass changes, of a single cell flowing therethrough. The mass change may be mass accumulation rate (MAR). When used with cancer cells, those changes provide a functional, universal biomarker by which medical professionals (e.g., oncologists) may monitor the progression of a cancer and determine how cancer cells respond to therapies.

The SMR may comprise an exquisitely sensitive scale that measures small changes in mass of a single cell. When cancer cells respond to cancer drugs, the cells begin the process of dying by changing mass within hours. The SMR can detect this minor weight change. That speed and sensitivity allow the SMR to detect a cancer cell's response to a cancer drug while the cell is still living. Upon flowing the live cells through the SMR, a functional biomarker, such as mass or MAR, in the one or more live cells is obtained. MAR measurements characterize heterogeneity in cell growth across cancer cell lines. Individual live cells are able to pass through the SMR, wherein each cell is weighed multiple times over a defined interval. The SMR includes multiple sensors that are fluidically connected, such as in series, and separated by delay channels. Such a design enables a stream of cells to flow through the SMR such that different sensors can concurrently weigh flowing cells in the stream, revealing single-cell MARs. The SMR is configured to provide real-time, high-throughput monitoring of mass change for the cells flowing therethrough. Therefore, the biophysical properties, including mass and/or mass changes (e.g., MAR), of a single cell can be measured. Such data can be stored and used in subsequent analysis steps, as will be described in greater detail herein.

Upon passing through the instrument 301, single cells remain viable and can be isolated downstream from the instrument 301 and are available to undergo the subsequent assays. As shown, a sample 209 of the one or more live cells having undergone the first assay (i.e., passing through the instrument 301) are collected in a suitable container 213 and are then available to undergo a second assay.

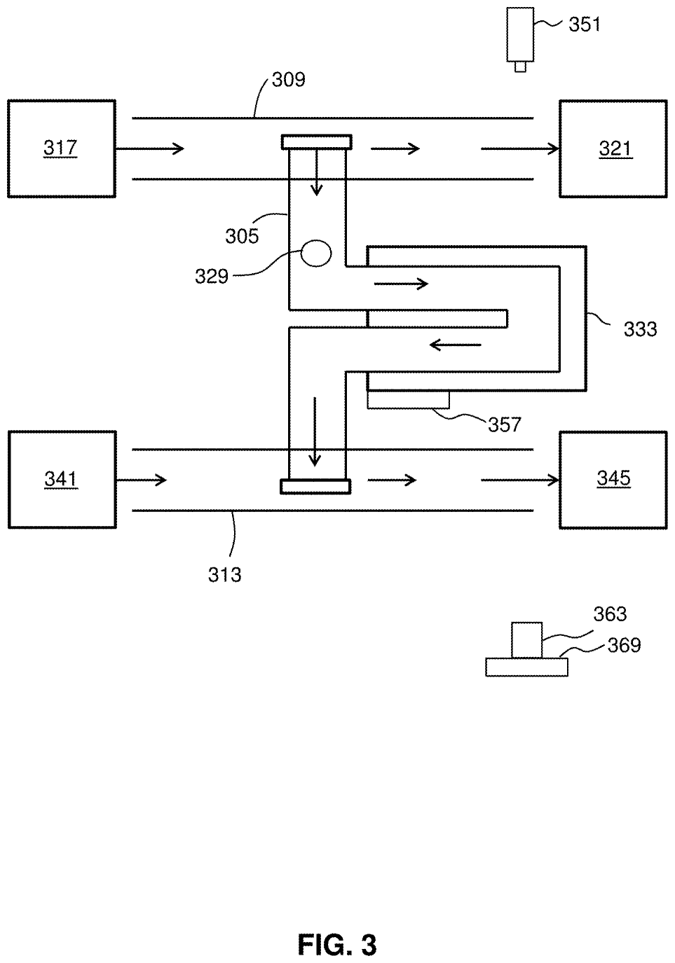

FIG. 3 shows a flow path through a suspended microchannel 305 of an SMR consistent with the present disclosure. As illustrated, the suspended microchannel 305 is suspended between an upper bypass channel 309 and a lower bypass channel 313. Having the two bypass channels allows for decreased flow resistance and accommodates the flow rate through the microchannel 305. Sample eluate 317 flows through the upper bypass channel 309, wherein a portion of the eluate 317 collects in the upper bypass channel waste reservoir 321. A portion of the eluate 317 including at least one live cell 329 flows through the suspended microchannel 305. The flow rate through the suspended microchannel 305 is determined by the pressure difference between its inlet and outlet. Since the flow cross section of the suspended microchannel is about 70 times smaller than that of the bypass channels, the linear flow rate can be much faster in the suspended microchannel than in the bypass channel, even though the pressure difference across the suspended microchannel is small. Therefore, at any given time, it is assumed that the SMR is measuring the eluate that is present at the inlet of the suspended microchannel. The sample includes a live cell or material with cell-like properties.

The cell 329 flows through the suspended microchannel 305. The suspended microchannel 305 extends through a cantilever 333 which sits between a light source 351 and a photodetector 363 connected to a chip 369 such as a field programmable gate array (FPGA). The cantilever is operated on by an actuator, or resonator 357. The resonator 357 may be a piezo-ceramic actuator seated underneath the cantilever 333 for actuation. The cell 329 flows from the upper bypass channel 309 to the inlet of the suspended microchannel 305, through the suspended microchannel 305, and to the outlet of the suspended microchannel 305 toward the lower bypass channel 313. A buffer 341 flows through the lower bypass channel towards a lower bypass channel collection reservoir 345. After the cell 329 is introduced to the lower bypass channel 313, the cell 329 is collected in the lower bypass collection reservoir 345.

In some embodiments, the instrument 301 comprises an array of SMRs with a fluidic channel passing therethrough.

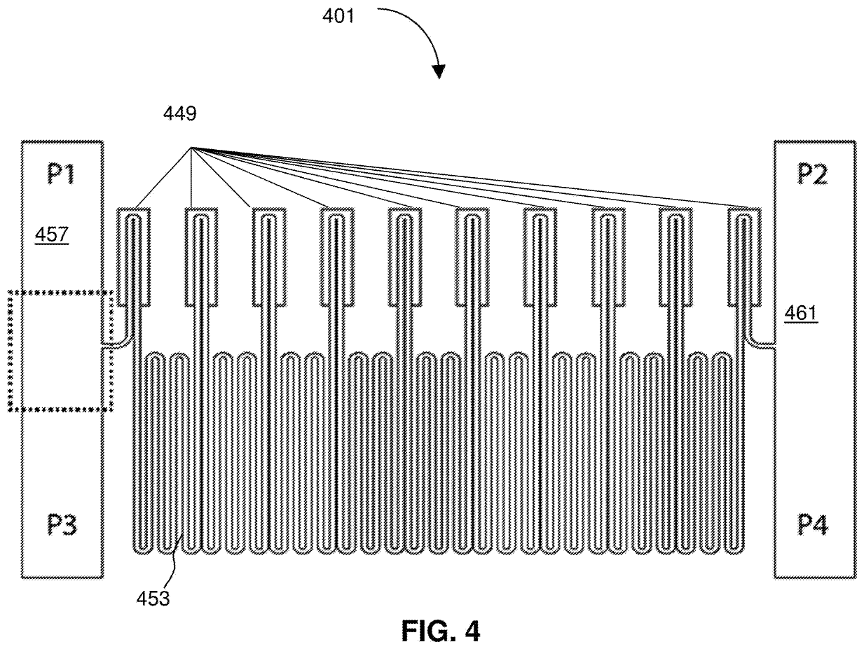

FIG. 4 shows a serial suspended microchannel resonator (sSMR) array 401, made up of an array of SMRs. An instrument that includes an sSMR array is useful for direct measurement of biophysical properties of single cells flowing therethrough. The sSMR includes a plurality of cantilevers 449 and a plurality of delay channels 453. Cells from the first bypass channel 457 through the cantilevers 449 and delay channels 453 to the second bypass channel 461. Pressure differences in the first bypass channel 457 are indicated by P1 and P2, and pressure differences in the second bypass channel 461 are indicated by P3 and P4.

Instruments 301 of the disclosure can make sensitive and precise measurements of mass or change in mass through the use of an sSMR array 401. The instruments use a structure such as a cantilever that contains a fluidic microchannel. Living cells are flowed through the structure, which is resonated and its frequency of resonation is measured. The frequency at which a structure resonates is dependent on its mass and by measuring the frequency of at which the cantilever resonates, the instrument can compute a mass, or change in mass, of a living cell in the fluidic microchannel. By flowing the isolated living cells from the tissue sample through such devices, one may observe the functions of those cells, such as whether they are growing and accumulating mass or not. The mass accumulation or rate of mass accumulation can be related to clinically important property such as the presence of cancer cells or the efficacy of a therapeutic on cancer cells.

Methods for measuring single-cell growth are based on resonating micromechanical structures. The methods exploit the fact that a micromechanical resonator's natural frequency depends on its mass. Adding cells to a resonator alters the resonator's mass and causes a measurable change in resonant frequency. Suspended microchannel resonators (SMRs) include a sealed microfluidic channel that runs through the interior of a cantilever resonator. The cantilever itself may be housed in an on-chip vacuum cavity, reducing damping and improving frequency (and thus mass) resolution. As a cell in suspension flows through the interior of the cantilever, it transiently changes the cantilever's resonant frequency in proportion to the cell's buoyant mass (the cell's mass minus the fluid mass it displaces). SMRs weigh single mammalian cells with a resolution of 0.05 pg (0.1% of a cell's buoyant mass) or better. The sSMR array 401 includes an array of SMRs fluidically connected in series and separated by “delay” channels between each cantilever 349. The delay channels give the cell time to grow as it flows between cantilevers.

Devices may be fabricated as described in Lee, 2011, Suspended microchannel resonators, Lab Chip 11:645 and/or Burg, 2007, Weighing of biomolecules, Nature 446:1066-1069, both incorporated by reference. Large-channel devices (e.g., useful for PBMC measurements) may have cantilever interior channels of 15 by 20 μm in cross-section, and delay channels 20 by 30 μm in cross-section. Small-channel devices (useful for a wide variety of cell types) may have cantilever channels 3 by 5 μm in cross-section, and delay channels 4 by 15 μm in cross-section. The tips of the cantilevers in the array may be aligned so that a single line-shaped laser beam can be used for optical-lever readout. The cantilevers may be arrayed such that the shortest (and therefore most sensitive) cantilevers are at the ends of the array. Before use, the device may be cleaned with piranha (3:1 sulfuric acid to 50% hydrogen peroxide) and the channel walls may be passivated with polyethylene glycol (PEG) grafted onto poly-L-lysine. In some embodiments, a piezo-ceramic actuator seated underneath the device is used for actuation. The instrument 301 may include low-noise photodetector, Wheatstone bridge-based amplifier (for piezo-resistor readout), and high-current piezo-ceramic driver. To avoid the effects of optical interference between signals from different cantilevers (producing harmonics at the difference frequency), the instrument may include a low-coherence-length light source (675 nm super-luminescent diode, 7 nm full-width half maximum spectral width) as an optical lever. After the custom photodetector converts the optical signal to a voltage signal, that signal is fed into an FPGA board, in which an FPGA implements twelve parallel second-order phase-locked loops which each both demodulate and drive a single cantilever. The FPGA may operate on a 100 MHz clock with I/O provided via a high-speed AD/DA card operating 14-bit analog-to-digital and digital-to-analog converters at 100 MHz.

To operate all cantilevers in the array, the resonator array transfer function is first measured by sweeping the driving frequency and recording the amplitude and phase of the array response. Parameters for each phase-locked loop (PLL) are calculated such that each cantilever-PLL feedback loop has a 50 or 100 Hz FM-signal bandwidth. The phase-delay for each PLL may be adjusted to maximize the cantilever vibration amplitude. The FM-signal transfer function may be measured for each cantilever-PLL feedback loop to confirm sufficient measurement bandwidth (in case of errors in setting the parameters). That transfer function relates the measured cantilever-PLL oscillation frequency to a cantilever's time-dependent intrinsic resonant frequency. Frequency data for each cantilever are collected at 500 Hz, and may be transmitted from the FPGA to a computer. The device may be placed on a copper heat sink/source connected to a heated water bath, maintained at 37 degrees C. The sample is loaded into the device from vials pressurized under air or air with 5% CO2 through 0.009 inch inner-diameter fluorinated ethylene propylene (FEP) tubing. The pressurized vials may be seated in a temperature-controlled sample-holder throughout the measurement. FEP tubing allows the device to be flushed with piranha solution for cleaning, as piranha will damage most non-fluorinated plastics. To measure a sample of cells, the device may initially flushed with filtered media, and then the sample may be flushed into one bypass channel. On large-channel devices, between one and two psi may be applied across the entire array, yielding flow rates on the order of 0.5 nL/s (the array's calculated fluidic resistance is approximately 3×10\hat{ }16 Pa/(m3/s). For small-channel devices, 4-5 psi may be applied across the array, yielding flow rates around 0.1 nL/s. Additionally, every several minutes new sample may be flushed into the input bypass channel to prevent particles and cells from settling in the tubing and device. Between experiments, devices may be cleaned with filtered 10% bleach or piranha solution.

For the data analysis, the recorded frequency signals from each cantilever are rescaled by applying a rough correction for the different sensitivities of the cantilevers. Cantilevers differing in only their lengths should have mass sensitivities proportional to their resonant frequencies to the power three-halves. Therefore each frequency signal is divided by its carrier frequency to the power three-halves such that the signals are of similar magnitude. To detect peaks, the data are filtered with a low pass filter, followed by a nonlinear high pass filter (subtracting the results of a moving quantile filter from the data). Peak locations are found as local minima that occur below a user-defined threshold. After finding the peak locations, the peak heights may be estimated by fitting the surrounding baseline signal (to account for a possible slope in the baseline that was not rejected by the high pass filter), fitting the region surrounding the local minima with a fourth-order polynomial, and finding the maximum difference between the predicted baseline and the local minima polynomial fit. Identifying the peaks corresponding to calibration particles allows one to estimate the mass sensitivity for each cantilever, such that the modal mass for the particles is equal to the expected modal mass.

Peaks at different cantilevers that originate from the same cell are matched up to extract single-cell growth information. The serial SMR array and can measure live cells.

Certain embodiments include devices with piezo-resistors doped into the base of each cantilever, which are wired in parallel and their combined resistance measured via a Wheatstone bridge-based amplifier. The resulting deflection signal, which consists of the sum of k signals from the cantilever array, goes to an array of k phase-locked loops (PLLs) where each PLL locks to the unique resonant frequency of a single cantilever. Therefore there is a one to one pairing between cantilevers and PLLs. Each PLL determines its assigned cantilever's resonant frequency by demodulating its deflection signal and then generates a sinusoidal drive signal at that frequency. The drive signals from each PLL are then summed and used to drive a single piezo actuator positioned directly underneath the chip, completing the feedback loop. Each PLL is configured such that it will track its cantilever's resonant frequency with a bandwidth of 50 or 100 Hz. After acquiring the frequency signals for each cantilever, the signals are converted to mass units via each cantilever's sensitivity (Hz/pg), which is known precisely.

Various embodiments of SMR and sSMR instruments, as well as methods of use, include those instruments/devices manufactured by Innovative Micro Technology (Santa Barbara, Calif.) and described in U.S. Pat. Nos. 8,418,535 and 9,132,294, the contents of each of which are hereby incorporated by reference in their entirety.

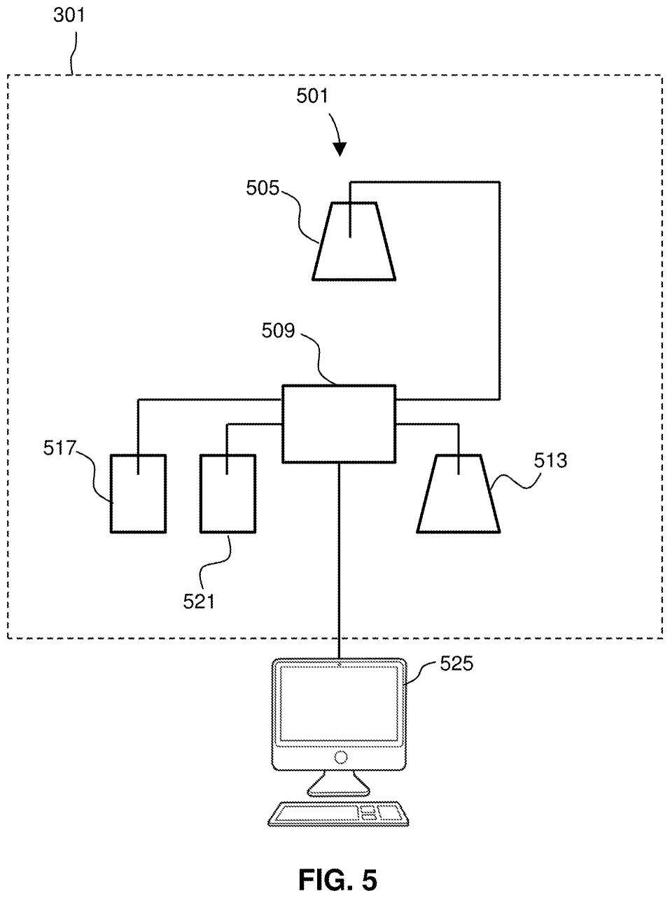

FIG. 5 shows a schematic diagram of an SMR detection system 501. As shown, a sample 505 (i.e., one or more live cells provided in a fluid medium) may be introduced to the SMR 509 of an instrument 301. As shown, the sample 505 and a buffer solution 513 may be provided to the SMR. The system 501 further includes an upper bypass channel waste outlet/reservoir 517 and lower bypass channel waste outlet/reservoir 521. The SMR 509 is configured to measure a functional biomarker of one or more live cells 505 flowing therethrough, such as density or mass of the sample, and transmit such measurements to a computer 525 that is communicatively coupled to the SMR 509, specifically communicatively coupled to the instrument 301. The computer 525 may be used for analysis and reporting of results. In some embodiments, a system for the functional biomarker measurement instrument may include additional analytical techniques, as will be described in greater detail herein. The computer 525 may further comprise a server and storage. Any of the elements in the SMR detection system 501 may interoperate via a network. The SMR 409 may include its own on-board computer. The computer 525 may include one or more processors and memory as well as an input/output mechanism.

Upon passing through the instrument 301, namely the exemplary flow path of a suspended microchannel or the flow path of the sSMR array 401, the cells remains viable and can be isolated downstream from the instrument 301 and are available to undergo the subsequent assays. The method further includes performing one or more additional assays on the live cells, either concurrently with the initial assay, or downstream from the first assay, to obtain further data associated with the live cells, such as additional functional data and/or genomic data.

It should be noted that methods of the disclosure include performing one or more additional assays on the live cells, either concurrently with the first assay, or downstream from the first assay, to obtain further functional or genetic data. In some embodiments, the second assay is performed on the live cells having undergone the first assay, which allows for data obtained from the first and second assays to be linked at a single-cell level, as opposed to a population level.

The mass accumulation rates (or other functional measurement) of cells from the patient sample in the presence of various therapeutic compounds (e.g., a chemotherapeutic), after exposure to various treatments (e.g., radiation), or combinations thereof can be compared to one another to select a next treatment for the patient. The various therapeutic compounds having been determined by the above in silico analysis based on suitability and/or likelihood of success for the individual patient, a practical comparison of efficacy as determined by effect on mass accumulation rate can be used to select a single treatment or combination thereof from the original list of suitable compounds or therapies. In certain embodiments, the mass accumulation rates may be compared to a minimal threshold and all compounds or treatments having mass accumulation rates below that threshold may be include in a report as options for treatment. In some embodiments, the compound, treatment, or combination thereof that resulted in the lowest mass accumulation rate may be selected for prescription or administration to the patient.

As previously described, after functional measurement, the one or more live cells may undergo a second assay to obtain further functional or genetic data. It should be noted that methods of the disclosure include performing one or more additional assays on the live cells, either concurrently with the first assay, or downstream from the first assay, to obtain further functional or genetic data. The second assay is performed on the live cells having undergone the first assay, which allows for data obtained from the first and second assays to be linked at a single-cell level, as opposed to a population level.

In some embodiments, the second assay is selected from the group consisting of genome sequencing, single cell transcriptomics, single cell proteomics, and single cell metabolomics. Genome sequencing is generally the process of determining the order of nucleotides in DNA. It includes any method or technology that is used to determine the order of the four bases: adenine, guanine, cytosine, and thymine. Single cell DNA genome sequencing involves isolating a single cell, performing whole genome amplification (WGA), constructing sequencing libraries, and then sequencing the DNA using a next-generation sequencer (e.g., Illumina, Ion Torrent, etc.). Single cell genome sequencing is particularly of interest in the field of cancer study, as cancer cells are constantly mutating and it is of great interest to observer how cancers evolve at the genetic level. For example, single cell genome sequencing allowing for patterns of somatic mutations and copy number aberration to be observed. Genome sequencing is described in greater detail herein.

Single-cell transcriptomics examines the gene expression level of individual cells in a given population by simultaneously measuring the messenger RNA (mRNA) concentration of hundreds to thousands of genes.

The purpose of single cell transcriptomics is to determine what genes are being expressed in each cell. The transcriptome is often used to quantify the gene expression instead of the proteome because of the difficulty currently associated with amplifying protein levels. Single-cell transcriptomics uses sequencing techniques similar to single cell genomics or direct detection using fluorescence in situ hybridization. The first step in quantifying the transcriptome is to convert RNA to cDNA using reverse transcriptase so that the contents of the cell can be sequenced using NGS methods, similar to what is done in single-cell genomics. Once converted, cDNA undergoes whole genome amplification (WGA), and then sequencing is performed. Alternatively, fluorescent compounds attached to RNA hybridization probes may be used to identify specific sequences and sequential application of different RNA probes will build up a comprehensive transcriptome.

Single cell transcriptomics can be used for various studies, such as, for example, gene dynamics, RNA splicing, and cell typing. Gene dynamics are usually studied to determine what changes in gene expression effect different cell characteristics. For example, this type of transcriptomic analysis has often been used to study embryonic development. RNA splicing studies are focused on understanding the regulation of different transcript isoforms. Single cell transcriptomics has also been used for cell typing, where the genes expressed in a cell are used to identify types of cells.

Single-cell proteomics is the study of proteomes (the entire complement of proteins that is or can be expressed by a cell, tissue, or organism) and their functions. The purpose of studying the proteome is to better understand the activity of cells at the single cells level. Since proteins are responsible for determining how the cell acts, understanding the proteome of single cell gives the best understanding of how a cell operates, and how gene expression changes in a cell due to different environmental stimuli. Although transcriptomics has the same purpose as proteomics it is not as accurate at determining gene expression in cells as it does not take into account post-transcriptional regulation.

There are three major approaches to single-cell proteomics: antibody based methods; fluorescent protein based methods; and mass-spectroscopy based methods. The antibody based methods use designed antibodies to bind to proteins of interest. These antibodies can be bound to fluorescent molecules such as quantum dots or isotopes that can be resolved by mass spectrometry. Since different colored quantum dots or different isotopes are attached to different antibodies it is possible to identify multiple different proteins in a single cell. Rare metal isotopes attached to antibodies, not normally found in cells or tissues, can be detected by mass spectrometry for simultaneous and sensitive identification of proteins. Another antibody based method converts protein levels to DNA levels. The conversion to DNA makes it possible to amplify protein levels and use NGS to quantify proteins. To do this, two antibodies are designed for each protein needed to be quantified. The two antibodies are then modified to have single stranded DNA connected to them that are complimentary. When the two antibodies bind to a protein the complimentary strands will anneal and produce a double stranded piece of DNA that can then be amplified using PCR. Each pair of antibodies designed for one protein is tagged with a different DNA sequence. The DNA amplified from PCR can then be sequenced, and the protein levels quantified.

In mass spectroscopy-based proteomics, there are three major steps needed for peptide identification: sample preparation; separation of peptides; and identification of peptides. Several groups have focused on oocytes or very early cleavage-stage cells since these cells are unusually large and provide enough material for analysis. Another approach, single cell proteomics by mass spectrometry (SCoPE-MS) has quantified thousands of proteins in mammalian cells with typical cell sizes (diameter of 10-15 μm) by combining carrier-cells and single-cell barcoding. Multiple methods exist to isolate the peptides for analysis. These include using filter aided sample preparation, the use of magnetic beads, or using a series of reagents and centrifuging steps. The separation of differently sized proteins can be accomplished by using capillary electrophoresis (CE) or liquid chromatograph (LC) (using liquid chromatography with mass spectroscopy is also known as LC-MS). This step gives order to the peptides before quantification using tandem mass-spectroscopy (MS/MS). The major difference between quantification methods is some use labels on the peptides such as tandem mass tags (TMT) or dimethyl labels which are used to identify which cell a certain protein came from (proteins coming from each cell have a different label) while others use not labels (quantify cells individually). The mass spectroscopy data is then analyzed by running data through databases that convert the information about peptides identified to quantification of protein levels. These methods are very similar to those used to quantify the proteome of bulk cells, with modifications to accommodate the very small sample volume. Improvements in sample preparation, mass-spec methods and data analysis can increase the sensitivity and throughput by orders of magnitude.

Single-cell metabolomics is study of chemical processes involving metabolites, the small molecule intermediates and products of metabolism, within cells. In particular, the purpose of single cell metabolomics is to gain a better understanding at the molecular level of major biological topics such as: cancer, stem cells, aging, as well as the development of drug resistance. In general the focus of metabolomics is mostly on understanding how cells deal with environmental stresses at the molecular level, and to give a more dynamic understanding of cellular functions. Accordingly, single cell metabolomics involves the study of a metabolome, which represents the complete set of metabolites in a biological cell, which are the end products of cellular processes. As generally understood, mRNA gene expression data and proteomic analyses reveal the set of gene products being produced in the cell, data that represents one aspect of cellular function. Conversely, metabolic profiling can give an instantaneous snapshot of the physiology of that cell, and thus, metabolomics provides a direct functional readout of the physiological state of an organism.

There are four major methods used to quantify the metabolome of single cells: fluorescence-based detection, fluorescence biosensors, FRET biosensors, and mass spectroscopy. The fluorescence-based detection, fluorescence biosensors, and FRET biosensors methods each use fluorescence microscopy to detect molecules in a cell. Such assays use small fluorescent tags attached to molecules of interest. However, it has been found that use of fluorescent tags may be too invasive for single cell metabolomics, and alters the activity of the metabolites. As such, the current solution to this problem is to use fluorescent proteins which will act as metabolite detectors, fluorescing whenever they bind to a metabolite of interest.

Mass spectroscopy is becoming the most frequently used method for single cell metabolomics, as there is no need to develop fluorescent proteins for all molecules of interest, and it is capable of detecting metabolites in the femto-mole range. Similar to the methods discussed in proteomics, there has also been success in combining mass spectroscopy with separation techniques such as capillary electrophoresis to quantify metabolites. Another method utilizes capillary micro-sampling combined with mass spectrometry and ion mobility separation, which has been demonstrated to enhance the molecular coverage and ion separation for single cell metabolomics.

In preferred embodiments, a second assay may include sequencing nucleic acid from the one or more live cells having undergone the first assay to produce sequence data. In order to perform nucleic acid sequencing, methods of the disclosure further include extracting nucleic acid from the one or more live cells having undergone the first analysis for a downstream sequencing step.

Isolation, extraction or derivation of genomic nucleic acids may be performed by methods known in the art. Isolating nucleic acid from a biological sample generally includes treating a biological sample in such a manner that genomic nucleic acids present in the sample are extracted and made available for analysis. Generally, nucleic acids are extracted using techniques such as those described in Green & Sambrook, 2012, Molecular Cloning: A Laboratory Manual 4 edition, Cold Spring Harbor Laboratory Press, Cold Spring Harbor, N.Y. (2028 pages), the contents of which are incorporated by reference herein. A kit may be used to extract DNA from tissues and bodily fluids and certain such kits are commercially available from, for example, BD Biosciences Clontech (Palo Alto, Calif.), Epicentre Technologies (Madison, Wis.), Gentra Systems, Inc. (Minneapolis, Minn.), and Qiagen Inc. (Valencia, Calif.). User guides that describe protocols are usually included in such kits.

It may be useful to lyse cells, isolate, and optionally amplify nucleic acid using methods known in the art. Amplification may be by polymerase chain reaction (PCR) as described in, e.g., Dieffenbach, PCR Primer, a Laboratory Manual, 1995, Cold Spring Harbor Press, Plainview, N.Y.; U.S. Pat. Nos. 4,683,195 and 4,683,202, all incorporated by reference. Nucleic acid isolation and optional purification provide a sample 209 that includes nucleic acid. The nucleic acid sample 209 may be subjected to an additional assay such as genomic sequencing.

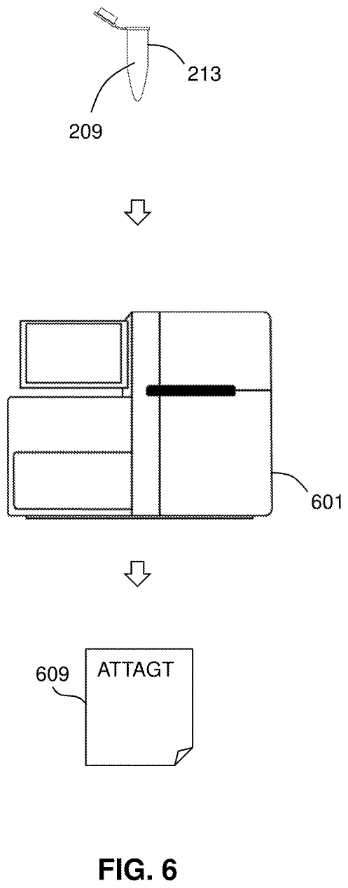

FIG. 6 illustrates performing an additional assay (i.e., sample 209 of cells collected from the device 301). Additional assays may include sequencing nucleic acid from the one or more live cells (from sample 209) using a sequencing instrument 601 to produce sequence data 609, and, in turn, methods of the invention may include analyzing 117 the sequence data 609 (as well as the measured cancer biomarker from the first assay). The sequence data 609 can be stored in databases of the invention along with associated patient, drug, and outcome information for future analysis in determining treatment options and predicting results.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, Illumina sequencing, or other so-called next generation sequencing techniques.

A sequencing technique that can be used includes, for example, Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which is incorporated by reference in their entirety.

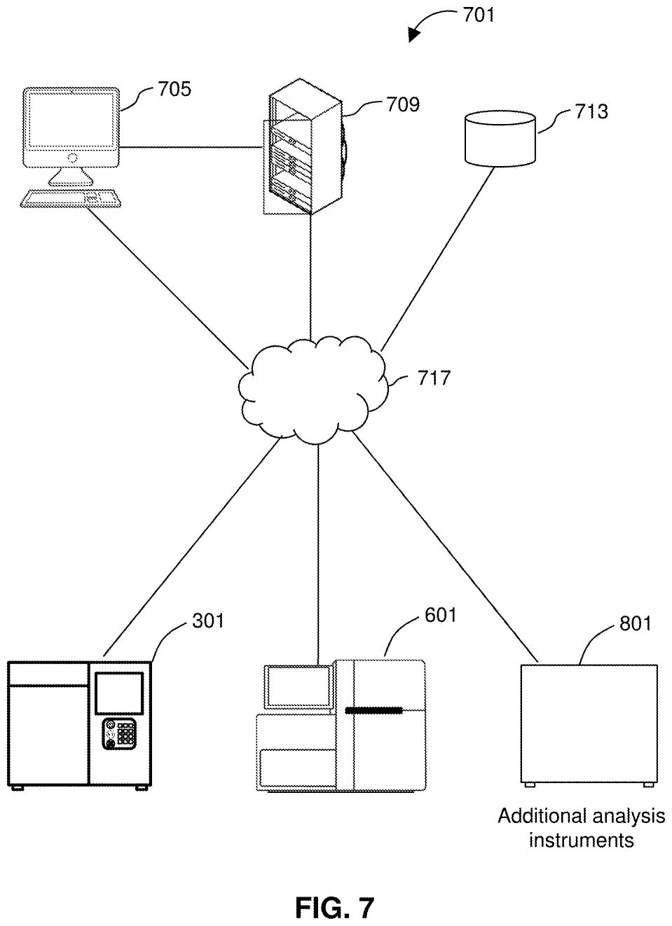

FIG. 7 is a block diagram of a system 701 according to embodiments of the invention. The system 701 may include one or more of an instrument 301 comprising a suspended microchannel resonator (SMR), a sequencing instrument 601, and any additional analysis instruments 801 for performing additional assays on the one or more cells downstream of the initial assay (performed by instrument 301), a computer 705, a server 709, and storage 713. Any of those elements may interoperate via a network 717. Any one of the instruments 301, 401, and 801 may include its own on-board computer. The computer 705 may include one or more processors and memory as well as an input/output mechanism. Where methods of the invention employ a client/server architecture, steps of methods of the invention may be performed using the server 709, which includes one or more of processors and memory, capable of obtaining data, instructions, etc., or providing results via an interface module or providing results as a file. The server 709 may be provided by a single or multiple computer devices, such as the rack-mounted computers sold under the trademark BLADE by Hitachi. The server 709 may be provided as a set of servers located on or off-site or both. The server 709 may be owned or provided as a service. The server 709 or the storage 713 may be provided wholly or in-part as a cloud-based resources such as Amazon Web Services or Google. The inclusion of cloud resources may be beneficial as the available hardware scales up and down immediately with demand. The actual processors—the specific silicon chips—performing a computation task can change arbitrarily as information processing scales up or down. In an embodiment, the server 709 includes one or a plurality of local units working in conjunction with a cloud resource (where local means not-cloud and includes or off-site). The server 709 may be engaged over the network 717 by the computer 705 and either or both may engage the outside database (not shown).

In system 701, each computer preferably includes at least one processor coupled to a memory and at least one input/output (I/O) mechanism. A processor will generally include a chip, such as a single core or multi-core chip, to provide a central processing unit (CPU). A process may be provided by a chip from Intel or AMD.

Memory can include one or more machine-readable devices on which is stored one or more sets of data or instructions (e.g., software) which, when executed by the processor(s) of any one of the disclosed computers can accomplish some or all of the methodologies or functions described herein. Memory may include one or more devices such as RAM, hard disks, solid-state memories (e.g., subscriber identity module (SIM) card, secure digital card (SD card), micro SD card, or solid-state drive (SSD)), optical and magnetic media, and/or any other tangible storage medium or media. A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

The system 701 or components of system 701 may be used to perform methods described herein. Instructions for any method step may be stored in memory and a processor may execute those instructions.

The system 701 thus includes at least one computer (and optionally one or more instruments) operable to obtain one or more live cells isolated from a sample of a patient, wherein the one or more live cells comprise at least one of a cancer cell and a cancer-related immune cell. Thus the invention provides a system with tools that may be used to inhibit the threat of relapse associated with minimal residual disease (MRD). Iterative MRD testing and treatment efficacy trials are used to continue to attack residual cancer cells even after treatment or remission. After treatments, patient samples may be taken periodically, and assayed on instrument 301 for minimal residual disease. In preferred embodiments, minimal residual diseases is detected using the instrument 301 to measure functional properties of living cells from the patient.

Once minimal residual disease is detected, the instrument 301 may be used to perform efficacy assays to determine effective therapies. The system provides for in vitro measurements of functional properties of individual living cells. Those properties may include mass or mass accumulation. The system 701 is further operable to perform a first assay via instrument 301 on the one or more live cells, wherein the first assay comprises measuring a functional cancer biomarker in the one or more live cells. The system 701 is further operable to perform a second assay via instrument 601 on the one or more live cells having undergone the first assay. The system 701 is further operable to analyze, by computer 705, data from the second assay and the measured cancer biomarker to determine at least a stage or progression of the cancer. Using the computer 701, the system is operable to provide a report comprising any suitable patient information including identity along with information related to the cancer evaluation, including, but not limited to, specific data associated with the first and second assays, a determination of a presence of MRD, and personalized treatment tailored to an individual patient's cancer.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for treating minimal residual disease, the method comprising:
obtaining a sample from a patient after cancer treatment;
conducting a first assay on the sample to detect minimal residual disease;
performing an assay to determine efficacy of a plurality of candidate therapies in treating the minimal residual disease; and
selecting an effective subsequent treatment for the patient from the plurality of candidate therapies.

2. The method of claim 1, wherein the first assay measures a functional feature of cells in the sample.

3. The method of claim 1, wherein the efficacy assay is performed in cancer cells obtained from the patient.

4. The method of claim 3, wherein the cells are live.

5. The method of claim 4, further comprising disaggregating the cells from a tissue sample before performing the efficacy assay.

6. The method of claim 4, wherein the efficacy assay comprises measuring a change in mass in the live cells.

7. The method of claim 6, wherein change in mass is measured using a suspended microchannel resonator instrument.

8. The method of claim 7, wherein the suspended microchannel resonator instrument comprises an array of suspended microchannel resonators.

9. The method of claim 8, wherein the suspended microchannel resonator instrument flows living cells through the array of suspended microchannel resonators.

10. The method of claim 6, wherein the suspended microchannel resonator instrument comprises a pressure control system operable to actively adjust fluidic pressure within the system to load living cells, one at a time, into a suspended microchannel resonator.

11. The method of claim 1, wherein the efficacy assay includes a functional measurement of live cells in the presence of one of the plurality of candidate treatments.

12. The method of claim 11, wherein the functional measurement comprises measuring a change in mass in the live cells.

13. The method of claim 1, further comprising administering the effective next treatment to the patient and repeating the conducting, performing, selecting and administering steps until the functional feature is not detected in the first assay.
